(19) United States
(12) Patent Application Publication
(10) Pub. No.: US 2024/0342108 A1
(43) Pub. Date: Oct. 17, 2024

(54) GREEN METHOD OF PRODUCING CIRCLE-SHAPED SILVER NANOPARTICLES USING ETHYL ACETATE EXTRACT OF URTICA DIOICA LEAVES

(71) Applicant: KING SAUD UNIVERSITY, RIYADH (SA)

(72) Inventors: SANDHANASAMY DEVANESAN, RIYADH (SA); MOHAMAD S. ALSALHI, RIYADH (SA); MOHAMMED AWADH BIN SALAH, RIYADH (SA); NASSER NOOH, RIYADH (SA)

(21) Appl. No.: 18/134,980

(22) Filed: Apr. 14, 2023

(51) Int. Cl. A61K 9/51; A61K 31/01; A61K 31/192; A61K 31/20; A61K 33/38; A61K 36/185; A61P 19/08

(52) U.S. Cl. CPC A61K 9/5192; A61K 31/01; A61K 31/192; A61K 31/20; A61K 33/38; A61K 36/185; A61P 19/08; A61K 2236/33; B82Y 5/00; B82Y 40/00

(57) ABSTRACT

A facile, simple route and green synthesis method of circle shaped silver nanoparticles using ethyl acetate extract of Urtica dioica leaves. The ethyl acetate extract was tested to identify the active components by using Gas chromatography-mass spectrometry [GC-MS]. 16 active derivatives were identified, of which three or four are considered as major components that are highly beneficial for providing bone regeneration, analgesic, anti-inflammatory and antimicrobial properties. The therapeutic bone regeneration components are TRIMETHYL-NEOPHYTADIENE, BENZENEDICARBOXYLIC ACID, and HEXADECANOIC ACID. The material interacts with a silver nitrate solution (AgNO₃) to produce silver nanoparticles (AgNPs). The AgNPs can be used for bone regeneration, analgesic, and anti-inflammatory purposes. This material is innovative and the biological benefits of this product report are entirely new in the scientific fields.

### BACKGROUND

### 1. Field

The disclosure of the present patent application relates to a green method of synthesizing circle shaped silver nanoparticles using ethyl acetate extract of Urtica dioica leaves, and particularly to the use of the such-produced silver nanoparticles to assist in bone regeneration.

### 2. Description of the Related Art

Urtica dioica, also known as the common nettle (Stinging nettle), is a perennial flowering plant in the family Urticaceae. Urtica dioica plants are spread out all over the world and have been a staple in herbal medicine since ancient times. The Urtica dioica plants contain several biological active compounds, which are capable of reducing inflammation, reducing blood sugars, and used to maintain sugar level in static conditions. While fresh nettle leaves may cause irritation, cooked, dried, or freeze-dried leaves are safe to consume. The medicinal values of nettle plant crude extract have been tested with several treatments, and have been reported to reduce capacity of bleeding, promote wound healing, serve as a natural diuretic and contain burn healing properties with animal models. Further, neck sores, infertility, lactation, abdominal pain, and internal injury have been generally known to be treated with nettle leaves, as disclosed by Pande, P. C. et al., “Ethnoveterinary plants of Uttaranchal—A review”, Ind. J. Trad. Know. (2007) 6, 444-458.

Another study showed the capacity to regenerate bone in calvarial defects of mice using a combination of Chitosan/dicarboxylic acid, which compounds were identified in the nettle leaves by Sukpaita T et al., “In Vivo Bone Regeneration Induced by a Scaffold of Chitosan/Dicarboxylic Acid Seeded with Human Periodontal Ligament Cells”, Int J Mol Sci, 2019 October 1; 20(19). Neophytadiene has demonstrated analgesic, antipyretic, anti-inflammatory, antimicrobial, and antioxidant properties, per Swamy MK1 et al., “GC-MS Based Metabolite Profiling, Antioxidant and Antimicrobial Properties of Different Solvent Extracts of Malaysian Plectranthus amboinicus Leaves”, Evid-Based Complement Alternat Med.

Thus, the synthesis of a composition including Urtica dioica leaves extract solving the aforementioned problems is desired.

### SUMMARY

The current subject matter relates to the synthesis of silver nanoparticles using an acetyl extract of Urtica dioica leaves and their biological benefits. The nanoparticles were confirmed by different characterization measurements. At the same time, biologically active compounds were identified by using GC- MS analysis. Three very active compounds in the extract have been identified. The innovative nanoparticles were tested with a variety of treatments.

In this regard, in one embodiment, the present subject matter relates to Urtica diocia silver nanoparticles comprising nanoparticles synthesized from Urtica diocia extract and silver. Also contemplated herein are pharmaceutical compositions comprising the Urtica diocia silver nanoparticles and a pharmaceutically acceptable carrier, as well as methods of providing a therapeutic effect to a subject, such as antimicrobial, analgesic, antipyretic, anti-inflammatory, antioxidant, wound healing, or homeostasis effects. Further included herein are methods for treating bone loss, or promoting bone regeneration, in a subject.

In another embodiment, the present subject matter relates to a method for obtaining an ethyl acetate Urtica diocia extract comprising: blinding cleaned and dried leaves of Urtica diocia to a fine powder; combining the fine powder with an ethyl acetate solvent to obtain a mixture; and obtaining the ethyl acetate Urtica diocia extract using a Soxlet extractor.

In an embodiment, the present subject matter relates to a silver nanoparticle composition including silver nanoparticles having a silver nanoparticle core and an organic layer coating over the core, the organic layer coating comprising Urtica diocia extract.

In a further embodiment, the present subject matter relates to a method of synthesizing Urtica diocia silver nanoparticles, comprising: dissolving silver nitrate in water to provide a silver solution; and adding the ethyl acetate Urtica diocia extract to the silver solution to provide a mixture including Urtica diocia silver nanoparticles. Urtica diocia silver nanoparticles prepared by this method are further contemplated herein.

These and other features of the present subject matter will become readily apparent upon further review of the following specification.

### BRIEF DESCRIPTION OF THE DRAWINGS

FIG. 1 is a graph showing a Gas Chromatography-Mass Spectrometry (GcMS) output of an ethyl acetate extract of Urtica dioica leaves.

FIG. 2 is a graph showing the UV-visible spectra for different intervals (8-24 hours) of nanoparticles synthesized using Urtica diocia leaves extract. The peak was observed at 420 nm, which is evidence of materials generated within 100 nm.

FIG. 3 is a graph showing FTIR spectral measurements of an ethyl acetate extract of Urtica dioica leaf.

FIGS. 4A and 4B are Scanning Electron Microscope images of silver nanoparticles formed using Urtica dioica leaves extract with different magnifications of 5 μm (4A) and 50 μm (43).

FIG. 5 is a graph showing the energy dispersive spectrum (EDX) of the synthesized silver nanoparticles formed using Urtica dioica leaves extract.

FIGS. 6A and 6B are Transmission Electron Microscope (TEM) images with different magnifications of 100 nm (6A) and 200 nm (6B) silver nanoparticles for Urtica dioica leaves.

FIG. 7 is a graph showing the Zeta potential image of Urtica dioica AgNPs.

FIG. 8 is a graph showing Zeta potential size distribution analysis of Urtica dioica AgNPs.

Similar reference characters denote corresponding features consistently throughout the attached drawings.

### DETAILED DESCRIPTION OF THE PREFERRED EMBODIMENTS

The following definitions are provided for the purpose of understanding the present subject matter and for construing the appended patent claims.

Throughout the application, where compositions are described as having, including, or comprising specific components, or where processes are described as having, including, or comprising specific process steps, it is contemplated that compositions of the present teachings can also consist essentially of, or consist of, the recited components, and that the processes of the present teachings can also consist essentially of, or consist of, the recited process steps.

It is noted that, as used in this specification and the appended claims, the singular forms “a”, “an”, and “the” include plural references unless the context clearly dictates otherwise.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

The term “optional” or “optionally” means that the subsequently described event or circumstance may or may not occur, and that the description includes instances where said event or circumstance occurs and instances in which it does not.

Unless defined otherwise, all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language. However, it will be understood by one of skill in the art, that in some specific instances, an embodiment can alternatively be described using the language “consisting essentially of” or “consisting of”.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

The current subject matter relates to the synthesis of silver nanoparticles using an acetyl extract of Urtica dioica leaves and their biological benefits. The nanoparticles were confirmed by different characterization measurements. At the same time, biologically active compounds were identified by using GC-MS analysis.

In this regard, in one embodiment, the present subject matter relates to Urtica diocia silver nanoparticles comprising nanoparticles synthesized from Urtica diocia extract and silver. In an embodiment in this regard, the Urtica diocia extract is an ethyl acetate extract. In other embodiments, the nanoparticles have an average particle diameter of about 8 nm to about 86 nm, an average particle diameter of about 25 nm to about 30 nm, or an average particle diameter of less than about 30 nm. In certain embodiments, a silver nanoparticle composition can include a Ag nanoparticle core coated with an organic layer comprising the Urtica diocia extract.

In certain embodiments, the Uritica diocia extract comprises one or more components selected from the group consisting of 2,3,6-TRIMETHYL-1,5-HEPTADIENE, 2-(2-BUTOXYETHOXY)-ETHANOL, FARNESOL, 3,7,11,15-TETRAMETHYL-2-HEXADECENE, 2,6,10-TRIMETHYL-NEOPHYTADIENE, 3,7,11,15-TETRAMETHYL-2-HEXADECANE, 1,2-BENZENEDICARBOXYLIC ACID, PHYTOL ACETATE, (E,E)-FARNESYLACETONE, 2,3,5,8-TETRAMETHYL-1,5,9-DECATRIENE, HEXADECANOIC ACID, 3,7,11,15-TETRAMETHYL-2-HEXADECEN-1-OL, [[(3.BETA.)-GORGOST-5-OL, LINOLENIC ACID, 1,2-BENZENEDICARBOXYLIC ACID, PALUSTROL, and a mixture or combination thereof. In this regard, the Urtica diocia extract, in some embodiments, comprises one or more components selected from the group consisting of 2,6,10-TRIMETHYL-NEOPHYTADIENE, HEXADECANOIC ACID, 1,2-BENZENEDICARBOXYLIC ACID, and a mixture or combination thereof, which are each effective for treating bone loss.

Also contemplated herein are pharmaceutical compositions comprising the Urtica diocia silver nanoparticles and a pharmaceutically acceptable carrier, as well as methods of providing a therapeutic effect to a subject, such as antimicrobial, analgesic, antipyretic, anti-inflammatory, antioxidant, wound healing, or homeostasis effects. Further included herein are methods for treating bone loss, or promoting bone regeneration, in a subject. In this regard, the present methods are effective for promoting bone regeneration in a subject.

In this regard, some of the present methods for treating bone loss in a subject can comprise administering to a subject in need thereof a therapeutically effective amount of the Urtica diocia silver nanoparticles (Urtica diocia-AgNPs) described herein for reducing bone loss, wherein the Urtica diocia silver nanoparticles can comprise one or more of HEXADECANOIC ACID, TRIMETHYL-NEOPHYTADIENE, and BENZENEDICARBOXYLIC ACID from the Urtica diocia extract, which are each effective for treating bone loss. Similarly, the present Urtica diocia silver nanoparticles can, in some embodiments, be useful to promote hemastosis, wound healing, bone production, and bone remodeling.

In another embodiment, the present subject matter relates to a method for obtaining an ethyl acetate Urtica diocia extract comprising: blinding cleaned and dried leaves of Urtica diocia as a fine powder; combining the fine powder with an ethyl acetate solvent to obtain a mixture; and obtaining the ethyl acetate Urtica diocia extract using a Soxlet extractor. In this regard, the mixture containing the blinded fine powder can be heating to its boiling point, for example, 77.1° C., by using the Soxlet extractor.

In a further embodiment, the present subject matter relates to a method of synthesizing Urtica diocia silver nanoparticles, comprising: dissolving silver nitrate (AgNO₃) in water to provide a silver solution; and adding the ethyl acetate Urtica diocia extract to the silver solution to provide a mixture including Urtica diocia silver nanoparticles. In certain embodiments in this regard, after the ethyl acetate is added to the Urtica diocia extract, an oxidation reduction reaction occurs for about 24 hours to obtain the Urtica diocia silver nanoparticles. In this regard, once the initiated oxidation and reduction process starts, the color of the mixture will typically change from clear to light brown, after about 8 hours or more, and it will typically completely turn dark brown after about 24 hours or more. Urtica diocia silver nanoparticles prepared by these methods are further contemplated herein. In this regard, the thus produced Urtica diocia silver nanoparticles can have a spherical shape when taken individually.

### EXAMPLES

### Example 1

Synthesis of Ethyl Acetate Extract of Urtica dioica

Urtica dioica (stinging nettle), a perennial plant belonging to the family of Urticaceae, genus Urtica, was collected from two different countries (Saudi Arabia, Riyadh Region and Tamil Nadu, Tanjore District, India). The fresh leaves were harvested in the field. The harvested leaves were cleaned and dried under laboratory temperature conditions. The dried leaves were collected and cleaned with running double distilled water and dried again under the same conditions. Finally, the completely cleaned and dried leaves were blinded as a fine powder.

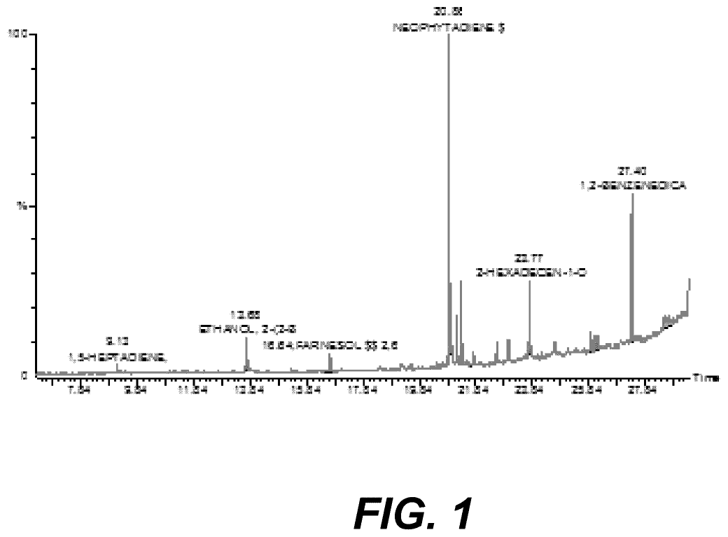

The blinded fine powder (20 gm) was flowed rapidly in 250 ml of ethyl acetate solvent, followed by heating to a boiling point of 77.1° C. by using a Soxlet extractor. The final product was collected and filtered with Whatman No. 1 filter paper. The purity purpose of the chemical constituents from the ethyl acetate extract of Urtica dioica was maintained, the dried compounds again washed with ethyl acetate, and the ethyl acetate was evaporated off. The final pure dried extract was collected, and the active constitutions from the ethyl acetate extract were identified by Gas chromatography-mass spectrometry (GC-MS). The GC-MS results found the chemical constitutions having the therapeutic properties of bone regeneration components are TRIMETHYL-NEOPHYTADIENE, (RT:20.86; area:19.590), ENZENEDICARBOXYLIC ACID, (RT: 27.40; area: 28.110), and HEXADECANOIC ACID (RT:23.04; area: 3.290), as shown in FIG. 1 and Table 1, below.

TABLE 1

| # | Name | RT | Area % | Area |
|---|---|---|---|---|
| 1 | 2,3,6-TRIMETHYL-1,5-HEPTADIENE | 9.13 | 2.230 | 53656 |
| 2 | 2-(2-BUTOXYETHOXY)-ETHANOL | 13.68 | 9.290 | 223326 |
| 3 | FARNESOL | 16.64 | 3.190 | 76594 |
| 4 | 3,7,11,15-TETRAMETHYL-2- | 20.77 | 0.630 | 15189 |
| | HEXADECENE | | | |
| 5 | 2,6,10-TRIMETHYL-NEOPHYTADIENE | 20.86 | 19.590 | 470716 |
| 6 | 3,7,11,15-TETRAMETHYL-2- | 21.14 | 2.010 | 48339 |
| | HEXADECANE | | | |
| 7 | 1,2-BENZENEDICARBOXYLIC ACID | 21.27 | 2.250 | 54076 |
| 8 | PHYTOL ACETATE | 21.36 | 6.370 | 153127 |
| 9 | (E,E)-FARNESYLACETONE | 21.77 | 2.110 | 50620 |
| 10 | ,2,3,5,8-TETRAMETHYL-1,5,9- | 22.62 | 2.540 | 61016 |
| | DECATRIENE | | | |
| 11 | HEXADECANOIC ACID | 23.04 | 3.290 | 79093 |
| 12 | 3,7,11,15-TETRAMETHYL-2- | 23.77 | 9.090 | 218363 |
| | HEXADECEN-1-OL | | | |
| 13 | [[(3.BETA.)-GORGOST-5-OL | 24.21 | 1.250 | 30044 |
| 14 | LINOLENIC ACID | 24.66 | 1.030 | 24719 |
| 15 | 1,2-BENZENEDICARBOXYLIC ACID | 27.40 | 28.110 | 675448 |
| 16 | PALUSTROL | 28.56 | 7.020 | 168638 |

### Example 2

### Synthesis of Silver Nanoparticles Using Ethyl Acetate Extract

1 mM silver nitrate was dissolved in 250 ml of double distilled water and 10 ml of ethyl acetate plant extract was added in the 1 mM silver nitrate solution and monitored for completion of an oxidation reduction process. The interaction of silver nitrate and plant extract was deeply monitored. The initiated oxidation and reduction process started (indicated by color changes) 8 h onwards and completely turned into dark brown within 24 h.

### Example 3

Characterization of Silver Nanoparticles Confirmation from Urtica dioica

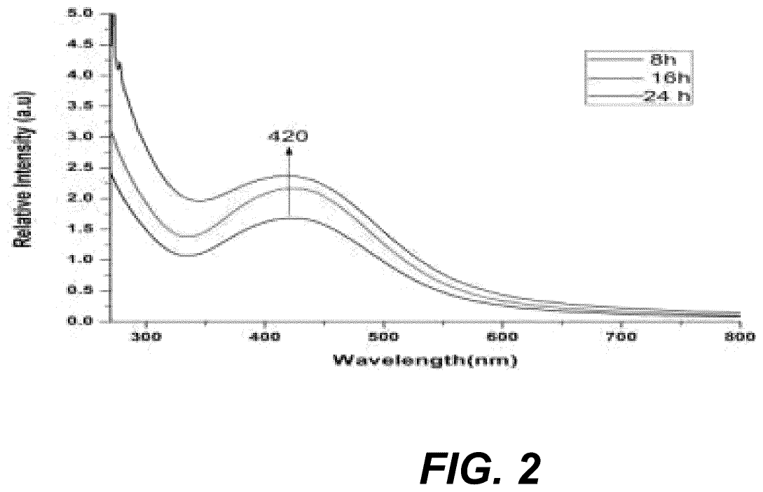

The UV-Visible spectra demonstrate the formation of silver nanoparticles from ethyl acetate extract of nettle plant leaves exhibit a characteristic absorption peak at 420 nm due to surface Plasmonic resonance (SPR), as seen in FIG. 2. The absorbance of the SPR band increases with different time (8-24 h) but the absorption values are slightly increased. This is evidence of the interaction between the AgNO₃ and ethyl acetate plant extract to form good silver nanoparticles.

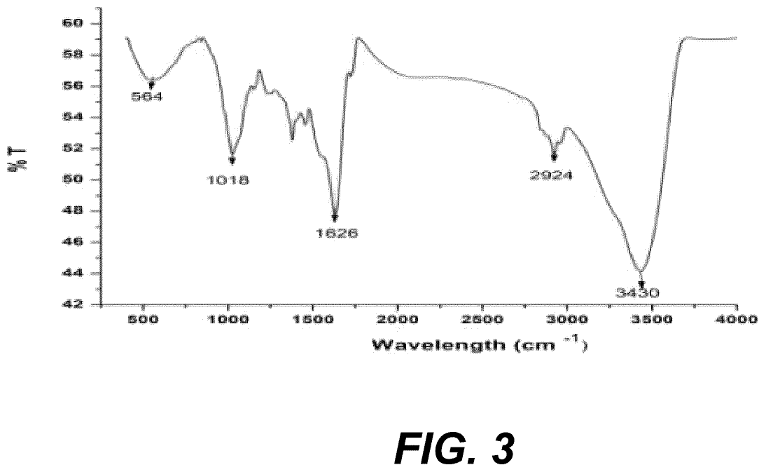

FTIR spectral measurements were carried out to identify the potential biomolecules in the ethyl acetate extract of the Urtica dioica leaf, which is used herein for reducing and capping the bioreduced AgNPs, as shown in FIG. 3. Common FTIR measurements were used to identify the presence of biomolecules which are mainly responsible for capping and stabilizing the metal nanoparticles synthesized by the ethyl acetate extract of the Urtica dioica leaf. The peaks at 564 cm⁻¹ correspond to C—Br stretching, exhibiting the presence of alkyl halides. Other exhibited prominent peaks at 1018, 1626, 2924 and 3430 cm⁻¹ were recorded from the ethyl acetate extract of Urtica dioica leaf silver nanoparticles. The FTIR spectra peaks are assigned aliphatic amines group; stretching vibration of (NH) C═O group; stretching vibration of C—H of alkane group and belonging to amide group and aromatic rings were considered as functional groups from flavonoids, triterpenoids, and polyphenols. The FTIR spectra provides evidence of the formation of silver nanoparticles from the ethyl acetate extract of Urtica dioica leaf extracts.

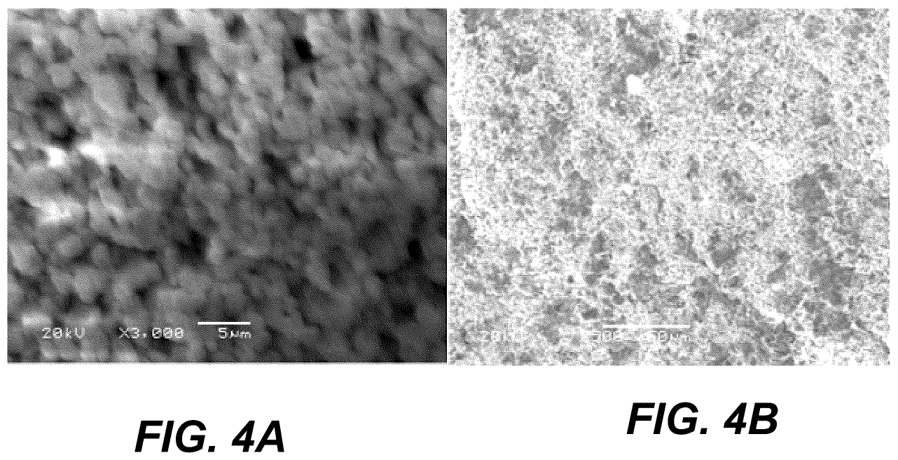

The surface morphology and size of the silver nanoparticles was analyzed by Scanning Electron Microscope (SEN) with different magnifications, 5 μm and 50 μm, in FIGS. 4A and 4B, respectively. The SEM images revealed single as well as several number of aggregated silver nanoparticles. It shows single nanoparticles are spherical in shape and aggregates were joined to each other to exhibit a larger shape in the morphology of synthesized silver nanoparticles.

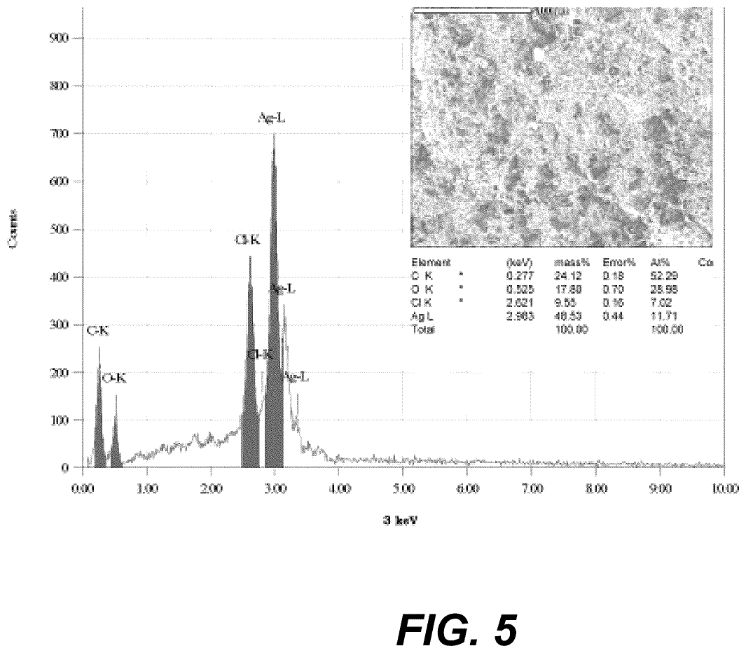

The energy dispersive spectrum (EDX) of the synthesized silver nanoparticles from Urtica dioica leaves extract is shown in FIG. 5. The EDX spectrum revealed the presence of silver as the ingredient element. Metallic AgNPs generally exhibit a typically strong signal peak at 3 keV, due to surface plasmon resonance. The EDX spectrum of FIG. 5 shows the quantitative measurement of biosynthesized AgNPs. The results reflect the presence of elements on a mass % basis, such as Ag (48,53%), O K (17.8%), C K (24.12%) and C1 (9,55%). The strong absorption peak in the region of 3 keV indicated that the product was composed of high purity Ag nanoparticles, indicating the formation of silver nanoparticles from Utica dioica leaves extract.

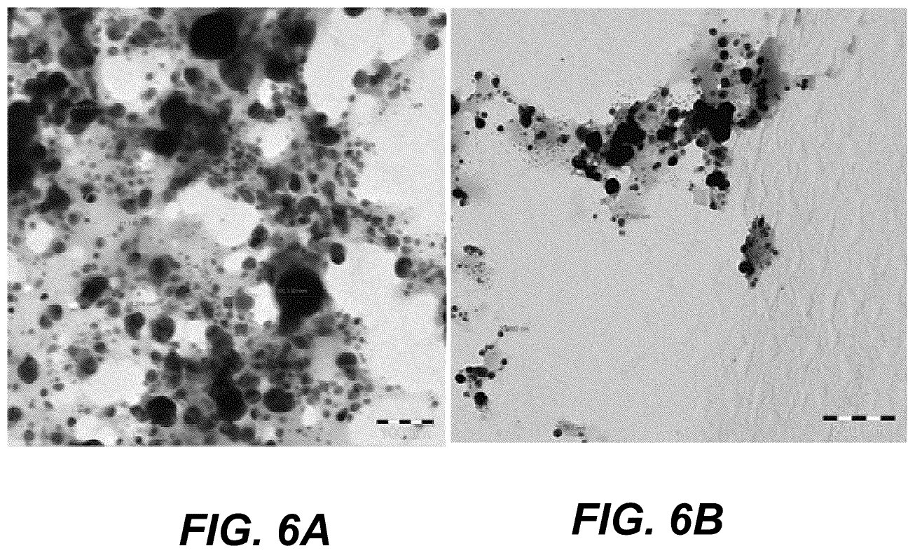

Transmission electron microscope (TEM) analysis with different magnifications of 100 nm and 200 nm are shown in FIGS. 6A and 6B, respectively. The images clearly indicate that the particles are spherical in shape. The synthesized particles are shown as well dispersed with a size of less than 100 nm. Mostly, the nanoparticles of the TEM images are not in physical contact or agglomerated with each other and are also separated with individual organic layers. Therefore, the TEM images clearly indicate the coating of Ag nanoparticles with an organic layer.

The nanoparticles disbursement details, including minimum, maximum, and average size are provided in Table 2 below.

TABLE 2

| Individual | | |
|---|---|---|
| measurement | 100 nm | 200 nm |
| 1 | 20.94 | 20.39 |
| 2 | 16.24 | 29.21 |
| 3 | 11.14 | 20.39 |
| 4 | 46.61 | 41.33 |
| 5 | 18.58 | 17.53 |
| 6 | 8.45 | 27.55 |
| 7 | 85.13 | 31.95 |
| Average | 29.584 | 26.90 |
| Minimum size | 8.45 | 17.53 |
| Maximum size | 85.13 | 41.33 |

The synthesis of silver nanoparticles achieved a minimal size of particles that are less than 20 nm and a maximum size of particles that are less than 100 nm. The average particle size was achieved at less than 30 nm, which was uniformly distributed without significant agglomeration.

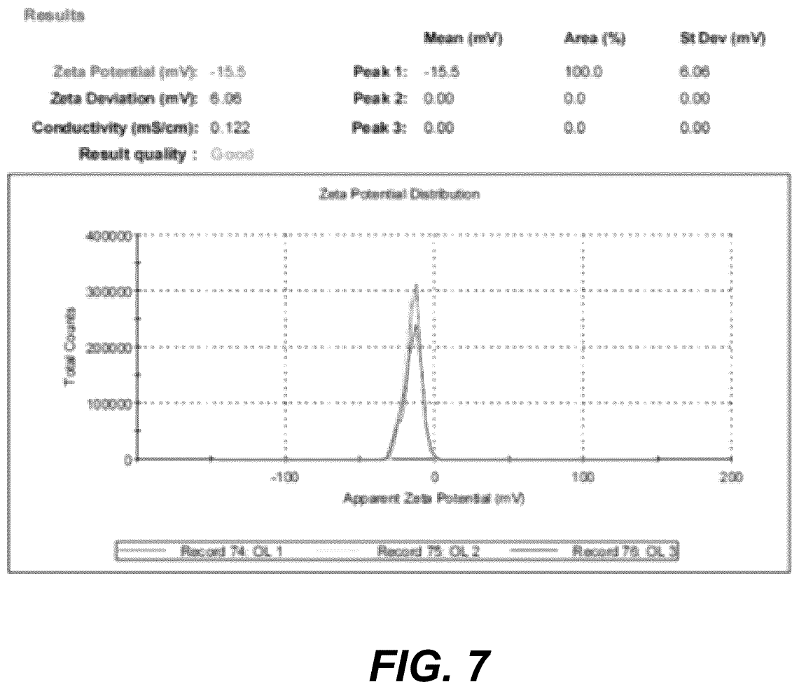

The Zeta potential image of Urtica dioica AgNPs is shown in FIG. 7. Zeta potential could enhance knowledge into the strength of the obtained AgNPs. The particles with zeta potential ranges falling in positive ranges less than +30 mV, or negative ranges more than −30 mV, are considered as stable particles. Accordingly, the present synthesized nanoparticles, having zeta potential values of negative 15.5 mV, are demonstrated to be stable.

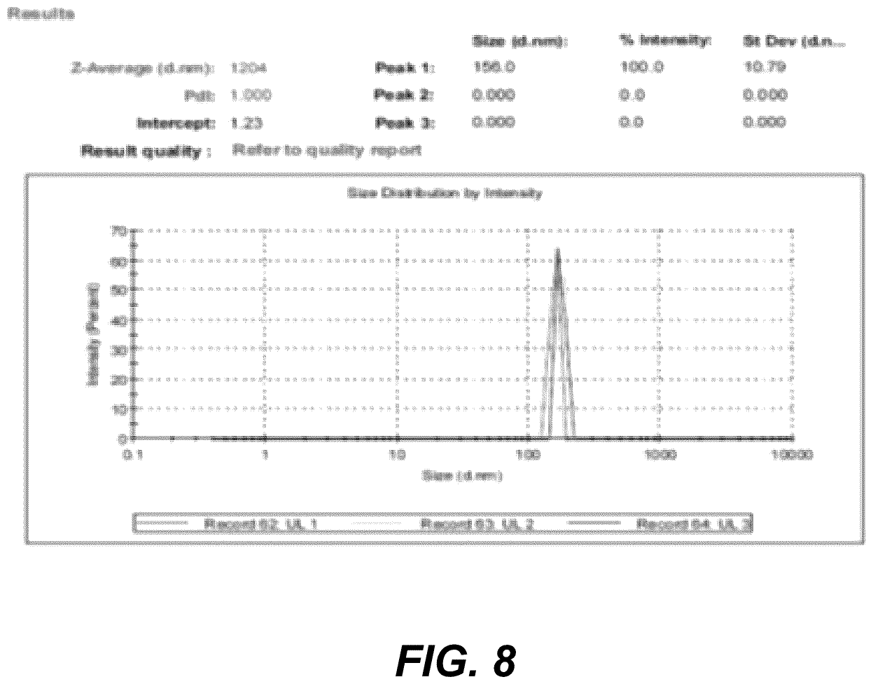

Zeta potential size distribution analysis was used to measure the dispersion of AgNPs from ethyl acetate leaves extract of Urtica dioica. The synthesized nanoparticles were shown to have the particle size distribution in FIG. 8.

Table 3, below, gives the information of the nanoparticle distribution with a less than 100 nm (35.94) size being achieved. The nano range of the AgNPs was given more effective fast recovery in wound healing.

TABLE 3

| Size (Xi) (d in | Number | | Average |
|---|---|---|---|
| nm) | (%)[Ni] | Ni*Xi | (Ni*Xi/Ni) |
| 21.04 | 0 | 0 | |
| 24.36 | 0 | 0 | |
| 28.21 | 0 | 0 | |
| 32.67 | 36.6 | 1195.722 | |
| 37.84 | 63.4 | 2399.056 | |
| 43.82 | 0 | 0 | |
| 50.75 | 0 | 0 | |
| 458.7 | 0 | 0 | |
| 531.2 | 0 | 0 | |
| 615.1 | 0 | 0 | |
| 712.4 | 0 | 0 | |
| 825 | 0 | 0 | |
| 955.4 | 0 | 0 | |
| 1106 | 0 | 0 | |
| | 100 | 3594.778 | 35.94778 |

### Example 4

### In Vivo Experimental Group and Surgical Procedure

The total number of male rats (1) was selected for an initial bone remodelling experiment. Before the experiments, the health of the rats was examined. The selected rat was healthy and very active with a body weight at 525 g and an age of 14 weeks. Then, a local aesthetic injection of Ketamine was injected to control the pain and movement during the removal of hair from the left thigh of the selected rat. Afterwards, the skin was cleaned with a gauze sponge containing 10% povidone-iodine solution to avoid any infections from microorganisms.

The initial protocol was completed and then the rat was anesthetized with Tekam 1 ml containing 50 mg ketamine, followed by a sevoflurane vaporizer with a single route channel for 20 minutes. The surgical site was approached through a 5 mm skin cut using a sterilized scalpel blade. The bleeding was swapped with a surgical sponge. After exposing the left thigh, the femur was cut with two holes of 3 mm in diameter.

All animal care and experimental procedures followed the guidance of the Experimental Surgery and Animal Laboratory and all animals were kept in individual cages with free access to food and water. The study was approved by the Institutional Animal Care and Use Committee of King Saud University (Approval No: SE-19-100), Riyadh, Saudi Arabia.

To measure the biological effects, cessation of bleeding, reduction of inflammation, and bone remodeling by the nanomaterials produced using ethyl acetate Urtica dioica leaves extract were monitored. 10 mg of the AgNPs was filled into the two femur bone defects and the opened skin was then stitched closed.

The rat survived the surgery without any complications, such as soft tissue damage at the defect site or stiffness of the adjacent knee and ankle joints. The rat was visually observed to be very active with healthy movement. There was no wound or bleeding seen at the defect site. The rat was deeply monitored every day, but it exhibited faster recovery and healthy movements. This provided evidence the present nanoparticles are capable of exhibiting wound healing properties, likely due to the phytoconstituents 3,7,11,15-TETRAMETHYL-2-HEXADECEN-1-OL; 2,6,10-TRIMETHYL-NEOPHYTADIENE; and 1,2-BENZENEDICARBOXYLIC ACID in the Urtica dioica leaves extract.

The rat was sacrificed exactly after the 7th week post-surgery and monitored for any inflammation in the upper part of the femur bone cut area, bone production (any soft tissue formation), and bone regeneration. Inflammation was found in the upper region of the femur bone, which was attached with the surgery bone. The inflammation was detached from the bone.

Another interesting effect was observed in the surgery bone. to which the new nanoparticles were applied. The nanoparticles interacted with blood flow from the cut bone, and they covered the entire cut bone and produced the soft tissue upper region of the bone. The soft cartilage tissue turned harder and mature or stronger bone occurred with bone remodeling in the rat.

Accordingly, the Urtica dioica AgNPs with active components adequately provided wound healing to enhance the blood circulation in the rat. The nanoparticles helped to enhance support for the recovery process, including hemastosis, wound healing, bone production, and bone remodeling.

The study was conducted over an at least 12 week period.

It is to be understood that the silver nanoparticles prepared using ethyl acetate extract of Urtica dioica leaves are not limited to the specific embodiments described above, but encompass any and all embodiments within the scope of the generic language of the following claims enabled by the embodiments described herein, or otherwise shown in the drawings or described above in terms sufficient to enable one of ordinary skill in the art to make and use the claimed subject matter.

1. (canceled)

2. (canceled)

3. (canceled)

4. (canceled)

5. (canceled)

6. A Urtica dioica silver nanoparticle composition, comprising nanoparticles having an Ag nanoparticle core and an organic layer coating over the core, the organic layer coating comprising Urtica dioica extract, wherein the nanoparticles have an average particle diameter of 35.94 nm.

7. The Urtica dioica silver nanoparticle composition of claim 6, wherein the Urtica dioica extract comprises one or more components selected from the group consisting of 2,3,6-trimethyl-1,5-heptadiene, 2-(2-butoxyethoxy)-ethanol, farnesol, 3,7,11,15-tetramethyl-2-hexadecene, 2,6,10-trimethyl-neophytadiene, 3,7,11,15-tetramethyl-2-hexadecane, 1,2-benzenedicarboxylic acid, phytol acetate, (e,e)-farnesylacetone, 2,3,5,8-tetramethyl-1,5,9-decatriene, hexadecanoic acid, 3,7,11,15-tetramethyl-2-hexadecen-1-ol, [[(3.beta.)-gorgost-5-ol, linolenic acid, 1,2-benzenedicarboxylic acid, palustrol, and a mixture or combination thereof.

8. The Urtica dioica silver nanoparticle composition of claim 7, wherein the Urtica dioica extract comprises one or more components selected from the group consisting of 2,6,10-trimethyl-neophytadiene, hexadecanoic acid, 1,2-benzenedicarboxylic acid, and a mixture or combination thereof, which are each effective for treating bone loss.

9. A pharmaceutical composition, comprising the Urtica dioica silver nanoparticle composition of claim 6 and a pharmaceutically acceptable carrier.

10. A method of providing a therapeutic effect to a subject, comprising administering to a subject in need thereof a therapeutically effective amount of the pharmaceutical composition of claim 9, wherein the therapeutic effect is selected from the group consisting of antimicrobial, analgesic, antipyretic, anti-inflammatory, antioxidant, wound healing, homeostasis, and combinations thereof.

11. A method for treating bone loss in a subject, comprising administering to a subject in need thereof a therapeutically effective amount of the pharmaceutical composition of claim 9 for reducing bone loss.

12. The method of claim 11, wherein administration of the pharmaceutical composition to the subject promotes bone regeneration in the subject.

13. A method for obtaining an ethyl acetate Urtica diocia extract comprising:
blinding cleaned and dried leaves of Urtica diocia as a fine powder;
combining the fine powder with an ethyl acetate solvent to obtain a mixture; and
obtaining the ethyl acetate Urtica diocia extract using a Soxlet extractor.

14. A method of synthesizing Urtica diocia silver nanoparticles, comprising:
dissolving silver nitrate in water to provide a silver solution; and
adding the ethyl acetate Urtica diocia extract of claim 13 to the silver solution to provide a mixture including Urtica diocia silver nanoparticles.

15. The method of claim 12, wherein after the ethyl acetate is added to the Urtica diocia extract, an oxidation reduction reaction occurs for about 24 hours to obtain the Urtica diocia silver nanoparticles.

16. Urtica diocia silver nanoparticles prepared by the method of claim 13.

17. A method for treating bone loss in a subject, comprising administering to a subject in need thereof a therapeutically effective amount of the Urtica diocia silver nanoparticles of claim 16 for reducing bone loss,
wherein the Urtica diocia silver nanoparticles comprise one or more of HEXADECANOIC ACID, TRIMETHYL-NEOPHYTADIENE, and BENZENEDICARBOXYLIC ACID from the Urtica diocia extract, each being effective for treating bone loss.

18. The method of claim 15, wherein the Urtica diocia silver nanoparticles promote hemastosis, wound healing, bone production and bone remodeling.
